(19) United States
(12) Patent Application Publication
(10) Pub. No.: US 2012/0322757 A1
(43) Pub. Date: Dec. 20, 2012

(54) Methods for Aloe Processing

(75) Inventors: Billy C. Coats, Garland, TX (US); Ivan E. Danhof, Grand Prairie, TX (US)

(73) Assignee: COATS ALOE INTERNATIONAL, INC., Dallas, TX (US)

(21) Appl. No.: 13/523,267

(22) Filed: Jun. 14, 2012

Related U.S. Application Data
(60) Provisional application No. 61/496,885, filed on Jun. 14, 2011.
(60) Provisional application No. 61/588,270, filed on Jan. 19, 2012.

(51) Int. Cl. A61K 31/122; A61K 31/715; A61P 33/00; A61P 37/02; A61P 35/00; A61P 33/06; A61K 31/704; A61P 1/10

(52) U.S. Cl. 514/54; 514/23; 514/680

(57) ABSTRACT

A composition and process for generating an anthraquinone rich aloe product from aloe leaves comprising the steps of: removing one or more rind parts from the aloe leaves comprising a top rind, a bottom rind, or both by filleting the aloe leaves on a filleting table into a mucilage layer below the rind parts of the aloe leaves to remove the top rind, the bottom rind, or both from an internal fillet or a gel layer; and separating the internal fillet or the gel layer from the rind parts; removing a mucilage layer attached to the one or more rind parts by scraping, heat-treating the rind parts and the extracted mucilage; and adding one or more preservatives to the heat-treated mucilage after cooling, wherein the extracted and heat-treated mucilage comprises the anthaquinone rich aloe product.

### CROSS-REFERENCE TO RELATED APPLICATIONS

The present application is a non-provisional application of U.S. Provisional Patent Application No. 61/496,885 filed Jun. 14, 2011 and U.S. Provisional Patent Application No. 61/588,270 filed Jan. 19, 2012, the entire contents of which are incorporated herein by reference.

### TECHNICAL FIELD OF THE INVENTION

The present invention relates in general to the field of manufacture of commercially valuable products from aloe vera, and more particularly, to a treatment method for the hand-filleted process of aloe vera leaves.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with Aloe vera processing.

U.S. Pat. No. 5,356,811 issued to Coats (1994) discloses a process for preparing a stabilized aloe vera gel which includes separating the clear gel from the whole leaf of the aloe vera plant, by grinding and mixing the whole leaf with a cellulose dissolving compound. Clear aloe vera gel is obtained through a series of filtration steps. Ultra violet light is used to sterilize the gel without the addition of extreme heat thereto. The material can be passed through a final organic filter to remove any remaining bacteria, and ultra-violet light is used to sterilize the gel without the addition of heat thereto. The material can be passed through a final organic filter to remove any remaining bacteria.

U.S. Pat. No. 6,083,508 issued to Avalos and Danhof (2000) relates to a process for forming an aloe product from only the leaf residue obtained after filleting aloe leaves having an internal fillet which is removed therefrom. Further, the residue is formed into a slurry by grinding and the aloe product is generated from the slurry. Hand filleting of the aloe leaves to remove the internal fillet may be performed as a preferable alternative to machine filleting. In addition the steps of preparing the aloe product comprises cleansing an aloe leaf before filleting it, separating the slurry formed into a liquid and solids, and further treating the separated liquid to remove laxatives before forming the aloe product. Also a process including all of the above steps may also be performed in order to form the liquid.

U.S. Pat. No. 5,925,357 issued to Cerqueira et al. (1999) discloses a bifurcated method to process an aloe whole leaf by processing the aloe fillet and the aloe rind separately and subsequently combining the products from these separate processes, and the use of the final combined product.

### SUMMARY OF THE INVENTION

The present invention describes a method of treating mucilage obtained from Aloe vera leaves during processing.

In one embodiment the instant invention describes a process for generating an anthraquinone rich aloe product from aloe leaves comprising the steps of: (i) removing one or more rind parts from the aloe leaves comprising a top rind, a bottom rind, or both comprising the steps of: a) filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer and b) separating the internal fillet or the gel layer from the rind parts; (ii) removing a mucilage layer attached to the one or more rind parts by scraping, heat-treating the rind parts, or by any other mechanical extraction techniques; (iii) heat-treating the extracted mucilage for a specified period of time; and (iv) adding one or more preservatives to the heat-treated mucilage after cooling, wherein the extracted and heat-treated mucilage comprises the anthaquinone rich aloe product. In one aspect of the method hereinabove, the extracted and heat-treated mucilage is further processed using one or more techniques selected from the group consisting of concentration, spray-drying, lyophilization, or any combinations thereof.

In another aspect the process of the instant invention further comprises the steps of: removing a base portion, a top portion and one or more spines along the margins of the aloe leaves and washing and cleaning the aloe leaves before filleting. In yet another aspect the process further comprise the step of collecting the mucilage accumulated on the filleting table, wherein the accumulated mucilage is added to the extracted mucilage prior to heat-treatment. In other related aspects extracted and heat-treated mucilage is enriched in one or more constituents comprising polysaccharides, organic acids, anthraquinones, and any combinations thereof and is assayed analytically for viscosity, total solids, calcium, magnesium, and malic acid. In another aspect the aloe leaves are derived from an Aloe species selected from the group consisting of Aloe vera, Aloe arborescens, Aloe aristata, Aloe dichotoma, Aloe nyeriensis, Aloe variegate, Aloe barbadensis, and Aloe wildii. In a specific aspect the aloe leaves are derived from Aloe barbadensis.

The step of filleting the aloe leaves as described in the above process is done by hand. The one or more preservatives used in the process above are selected from the group consisting of benzoic acid, potassium sorbate, and/or other preservatives approved for human consumption by, e.g., the Federal Food and Drug Administration or equivalent regulatory agency. In one aspect the internal fillet or gel is processed by one or more techniques to produce a medicinal composition for topical application, oral administration, or parenteral administration for providing immunostimulation, immunomodulation, cancer treatment, diabetes treatment, anti-inflammatory action, or any combinations thereof. In another aspect the anthraquinone rich aloe product may be combined with one or more medicinal preparations prepared from the internal fillet or the gel. Finally, the instant invention describes an anthraquinone rich aloe product generated by the process described hereinabove.

Another embodiment of the instant invention relates to an anthraquinone rich composition comprising: a concentrated liquid, a lyophilized powder, a spray dried powder, or any combinations thereof comprising heat-treated and extracted mucilage from one or more rind parts from aloe leaves, wherein the composition comprises one or more preservatives. In one aspect of the composition disclosed herein the heat-treated and extracted mucilage is generated by a process comprising the steps of: (i) removing one or more rind parts from the aloe leaves comprising a top rind, a bottom rind, or both comprising the steps of: a) filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer and b) separating the internal fillet or the gel layer from the rind parts; (ii) removing a mucilage layer attached to the one or more rind parts by scraping, heat-treating the rind parts, or by any other mechanical extraction techniques; (iii) heat-treating the extracted mucilage for a specified period of time; and (iv) adding one or more preservatives to the heat-treated mucilage after cooling.

The process described hereinabove further comprises the steps of: (i) removing a base portion, a top portion and one or more spines along the margins of the aloe leaves and (ii) washing and cleaning the aloe leaves before filleting. In one aspect the process further comprise the step of collecting the mucilage accumulated on the filleting table, wherein the accumulated mucilage is added to the extracted mucilage prior to heat-treatment. In another aspect the step of filleting the aloe leaves is done by hand. In yet another aspect the internal fillet or gel is processed by one or more techniques to produce a medicinal composition for topical application, oral administration, or parenteral administration. In a related aspect the extracted and heat-treated mucilage is enriched in one or more constituents comprising polysaccharides, organic acids, anthraquinones, and any combinations thereof. In one aspect the composition is used for a treatment of one or more conditions selected from constipation, malaria, cancer, or any combinations thereof. In another aspect the one or more preservatives are selected from the group consisting of benzoic acid, potassium sorbate, and/or other preservatives approved for human consumption by, e.g., the Federal Food and Drug Administration or equivalent regulatory agency.

Yet another embodiment described herein relates to a method of treating constipation, malaria, or any other parasitic infection in a human subject comprising the steps of: (i) identifying the human subject in need of treatment against the constipation, malaria or any other parasitic infection and (ii) administering a pharmaceutical composition comprising an anthraquinone rich aloe product to the human subject in an amount effective to treat the constipation, malaria or any other parasitic infection, wherein the composition comprises heat-treated and extracted mucilage from one or more rind parts from aloe leaves along with one or more preservatives.

The pharmaceutical composition described hereinabove is made by a process comprising the steps of: 1) removing one or more rind parts from the aloe leaves comprising a top rind, a bottom rind, or both comprising the steps of: filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer and separating the internal fillet or the gel layer from the rind parts, 2) removing a mucilage layer attached to the one or more rind parts by scraping, heat-treating the rind parts, or by any other mechanical extraction techniques, 3) heat-treating the extracted mucilage for a specified period of time, and 4) adding one or more preservatives to the heat-treated mucilage after cooling. In one aspect the composition is adapted for topical, oral, or parenteral administration.

A pharmaceutical composition for treatment of constipation, malaria or any other parasitic infection in a human subject is described in one embodiment of the present invention.

The composition as described herein comprises an anthraquinone rich aloe product, wherein the composition comprises heat-treated and extracted mucilage from one or more rind parts from aloe leaves along with one or more preservatives.

Another embodiment of the present invention provides a method of providing immunostimulation or immunomodulation, treating cancer or any combinations thereof in a human subject comprising the steps of: identifying the human subject in need of immunostimulation, immunomodulation, treatment of the cancer, or any combinations thereof; and administering a pharmaceutical composition comprising an anthraquinone rich aloe product to the human subject in an amount effective to provide immunostimulation, immunomodulation, treatment of the cancer, or any combinations thereof, wherein the composition comprises heat-treated and extracted mucilage from one or more rind parts from aloe leaves along with one or more preservatives and is administered to the human subject orally or parenterally.

The pharmaceutical composition disclosed in the method hereinabove is made by a process comprising the steps of: removing one or more rind parts from the aloe leaves comprising a top rind, a bottom rind, or both comprising the steps of: filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer; and separating the internal fillet or the gel layer from the rind parts; removing a mucilage layer attached to the one or more rind parts by scraping, heat-treating the rind parts, or by any other mechanical extraction techniques; heat-treating the extracted mucilage for a specified period of time; and adding one or more preservatives to the heat-treated mucilage after cooling.

Finally, the present invention discloses a pharmaceutical composition for providing immunostimulation or immunomodulation, treating cancer or any combinations thereof in a human subject comprising: an anthraquinone rich aloe product, wherein the composition comprises heat-treated and extracted mucilage from one or more rind parts from aloe leaves along with one or more preservatives.

### BRIEF DESCRIPTION OF THE DRAWINGS

For a more complete understanding of the features and advantages of the present invention, reference is now made to the detailed description of the invention along with the accompanying figures and in which:

FIG. 1 is a proton-nuclear magnetic resonance profile showing the different components of untreated (raw) mucilage obtained from Aloe vera; and

FIG. 2 is a proton-nuclear magnetic resonance profile showing the different components of treated (heat) mucilage obtained from Aloe vera.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The term “Aloe” refers to the genus of South African plants of the Liliaceae family of which the Aloe barbadensis plant is a species.

The present invention describes a novel process for treating the mucilage layer obtained from the leaf rind or the mucilage which gets accumulated on top of the filleting table during the traditional, labor-intensive hand-filleting method for processing aloe leaves.

The mucilage layer that is obtained or which is accumulated during the processing of the Aloe leaves is typically discarded. However, the method of the present invention provides a process for producing a product from the discarded mucilage that contains desirable constituents in quantities in excess of what can be obtained from traditional processing methods.

Aloe vera is a subtropical plant which has elongated leaves containing a clear, viscous gel. The leaves are given structural rigidity by means of a plurality of hair-like connective tubules (vascular bundles) which run longitudinally from tip to base of the leaf. The plant is the source of at least two medicinal substances which includes a mucilaginous yellow fluid which can be drained from the connective tubules of the vascular bundles at the base of the leaves adjacent to the leaf rind. This yellow fluid contains the aloe anthraquinones, which include aloin A (barbaloin), aloin B (isobarbaloin), aloe-emodin, and emodin, which can be used as a cathartic or a medicinal purge. The mucliage, located between the thick outer green rind and the central fillet, accumulates on the filleting table top as the thick green rinds are removed. This material is typically discarded during the processing of the Aloe vera leaves. The other medicinal substance is the clear gel taken from the centrally-located internal fillet of the elongated leaf and which has been used throughout history for its medicinal and therapeutic healing actions.

The aloe leaf consists of three layers. The first is the thick outer green rind. The second is a viscous, jelly-like mucilage layer into which vascular bundles, attached to the inner surface of the rind, protrude. The third is the fillet proper which has a structural integrity consisting of hexagonal structures containing the fillet fluid.

During the growth of the aloe plant, the materials of the mucilage layer, subsequent to their synthesis, are distributed to the storage cells (cellulose-reinforced hexagons) of the fillet, a process which is accompanied by dilution owing to the water which is stored in the fillet cells. The fillet consists of about 98% water. The pericyclic cells located at the top of the vascular bundles contain a yellow liquid called “yellow sap” or “latex”. This material contains high concentrations of aloin and similar anthraquinones that exert a powerful laxative action when taken internally.

In order to avoid contaminating the internal fillet with the yellow sap, traditional hand-filleting is the method of choice for processing aloe leaves. In this method, the lower one inch of the leaf base, the tapering point (2-4 inches) of the leaf top, and the short, sharp spines located along the leaf margins are removed by a sharp knife. The knife is then introduced into the mucilage layer below the green rind to avoid the vascular bundles and the top rind is removed. The bottom rind is similarly removed and the rind parts, to which a significant amount of mucilage remains attached, are discarded. Another portion of the mucilage layer is accumulated on top of the filleting table and is also discarded. As an additional procedure to limit inclusion of the inadvertent laxative anthraquinones, the fillets may be washed in water removing a majority of the deep layer of mucilage attached to the outer surface of the structurally integral fillet.

A second method of processing aloe leaves was also developed that uses the whole leaf and is called the “whole leaf aloe process” as disclosed in U.S. Pat. No. 6,083,508 incorporated herein by reference. In this process, the base, tip, and lateral spines of the aloe leaf are removed as previously stated leaving intact the thick outer green rinds. The leaf is then cut into sections and ground into a particulate slurry material. The slurry is then treated with special chemical products that break down the hexagonal structure of the fillet releasing the constituents. By using a series of coarse screening filters or passage through a juice press, the rind particles are removed. The remaining juice is then passed through various filtering columns which remove the undesirable laxative agents. This process, performed properly, can produce a constituent-rich juice virtually free of the laxative anthraquinones. This process, developed in the 1980's, is considerably less labor intensive and is more cost effective. However, in this process, the large amount of water in the fillet in the leaf (50%+) gets processed and dilutes the valuable constituents in the aloe product.

In order to increase the desirable constituents of processed aloe in the final product, the elements in the product may be concentrated by (1) heat, (2) heat and vacuum, and (3) reverse osmosis. Variously processed aloe juices can be reduced to powder form which improves shelf life compared to liquid products and eliminates the cost of shipping water. In the spray-dried powder process, the liquid aloe is sprayed onto a matrix, usually high molecular weight maltodextrins, that usually constitute about 50% or more of the final product, using high heat. The high heat exposure changes to some degree some of the potentially beneficial constituents. The lyophilized or freeze-dried powder utilizes cold (about −85° C.) and vacuum (usually about one-third atmosphere) which causes evaporation and sublimation of only the water. Heat-induced changes in the beneficial constituents are avoided but the process is considerably more expensive than the spray-dried process.

In the third process, fillets of aloe can be reduced to dehydrated pellets by placing them in a commercial-scale vegetable dehydrator operated at relatively low temperatures (slightly above body temperature) but for many hours. The dehydrated pellets are then ground to a fine powder.

The constituents that are most constant in the aloe plant and that are used as a standard by which to judge an aloe product are (1) total solids, (2) calcium, (3) magnesium, and (4) malic acid. The standard for calcium in the hand-filleted product averages 241 milligrams per liter. The standard for magnesium in the hand-filleted product averages 58.4 milligrams per liter. The standard for malic acid in the hand-filleted product averages 2,028 milligrams per liter. Finally, the standard for solids in the hand-filleted aloe product averages 0.83 percent.

It is known that the discarded residue from the filleting process that includes the rind contains a significant amount of mucilage that remains attached. It has been discovered that the highest concentration of potentially beneficial aloe constituents are found in this mucilage along with the rind as this layer represents the leaf constituents synthesized by the vascular bundle cells and powered by the energy developed in the green chlorophyll-containing rind cells through sun-induced photosynthesis.

It would be advantageous to make use of the discarded residue of the filleted process to have an aloe product that contains an enviably high concentration of desirable constituents that are virtually free of undesirable laxative anthraquinones. The novel process of the present invention addresses this issue and produces a product containing desirable constituents that may exceed those of aloe products subjected to concentration procedures intended to increase these desirable constituents.

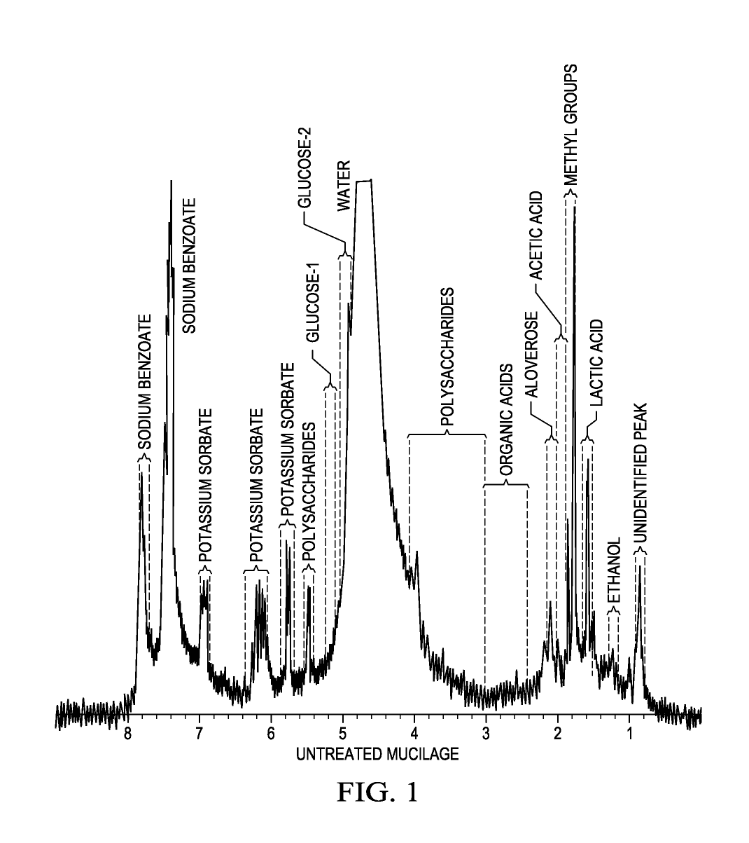

FIG. 1 is a p-NMR (proton-nuclear magnetic resonance) profile showing the different components of the untreated (raw) mucilage obtained from Aloe vera. The two sodium benzoate peaks are present as are the three potassium sorbate peaks followed by the small polysaccharide peak and the glucose-1 and glucose-2 peaks. The water peak is prominent.

The typical three major polysaccharide peaks are entirely absent as are the organic acids apart from the early lactic acid peak following the relatively small alverose and acetic acid peaks interspersed with methyl groups. A relatively small ethanol peak is followed by an unidentified peak at 0.8 ppm.

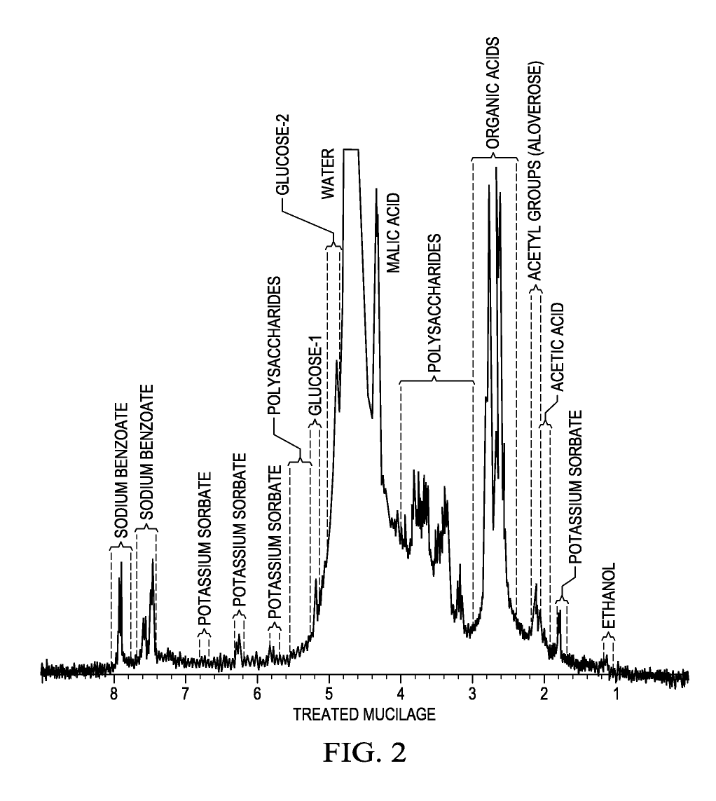

FIG. 2 is a p-NMR (proton-nuclear magnetic resonance) profile showing the different components of heat-treated mucilage obtained from Aloe vera. The principal sodium benzoate peaks are identified as are the three potassium sorbate peaks. Then encountered are the small polysaccharide peak and the glucose-1 and glucose-2 peaks.

The three classical major polysaccharide peaks (small, medium, and large molecular species) are prominently seen. Organic acids are well constituted. The aloverose and acetic acid peaks are identified and followed by a small potassium sorbate peak and very small ethanol peak.

It is contemplated that any embodiment discussed in this specification can be implemented with respect to any method, kit, reagent or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 5,356,811: Method of Processing Stabilized Aloe vera Gel Obtained from the Whole Aloe vera Leaf.
U.S. Pat. No. 6,083,508: Method of Processing Aloe Leaves.
U.S. Pat. No. 5,925,357: Bifurcated Method to Process Aloe Whole Leaf.

What is claimed is:

1. A process for generating an anthraquinone rich aloe product from aloe leaves comprising the steps of:
removing one or more rind parts from the aloe leaves comprising a top rind, a bottom rind, or both comprising the steps of:
filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer; and
separating the internal fillet or the gel layer from the rind parts;
removing a mucilage layer attached to the one or more rind parts by scraping, heat-treating the rind parts, or by any other mechanical extraction techniques;
heat-treating the extracted mucilage for a specified period of time; and
adding one or more preservatives to the heat-treated mucilage after cooling, wherein the extracted and heat-treated mucilage comprises the anthaquinone rich aloe product.

2. The process of claim 1, wherein the extracted and heat-treated mucilage is further processed using one or more techniques selected from the group consisting of concentration, spray-drying, lyophilization, or any combinations thereof.

3. The process of claim 1, wherein the process further comprises the steps of:
removing a base portion, a top portion and one or more spines along the margins of the aloe leaves; and
washing and cleaning the aloe leaves before filleting.

4. The process of claim 1, wherein the process further comprise the step of collecting the mucilage accumulated on the filleting table, wherein the accumulated mucilage is added to the extracted mucilage prior to heat-treatment.

5. The process of claim 1, wherein the extracted and heat-treated mucilage is enriched in one or more constituents comprising polysaccharides, organic acids, anthraquinones, and any combinations thereof.

6. The process of claim 1, wherein the extracted and heat-treated mucilage is assayed analytically for viscosity, total solids, calcium, magnesium, and malic acid.

7. The process of claim 1, wherein the aloe leaves are derived from an Aloe species selected from the group consisting of Aloe vera, Aloe arborescens, Aloe aristata, Aloe dichotoma, Aloe nyeriensis, Aloe variegate, Aloe barbadensis, and Aloe wildii.

8. The process of claim 1, wherein the aloe leaves are derived from Aloe barbadensis.

9. The process of claim 1, wherein the step of filleting the aloe leaves is done by hand.

10. The process of claim 1, wherein the one or more preservatives are selected from the group consisting of benzoic acid, potassium sorbate, and/or other preservatives approved for human consumption.

11. The process of claim 1, wherein the internal fillet or gel is processed by one or more techniques to produce a medicinal composition for topical application, oral administration, or parenteral administration.

12. The process of claim 11, wherein the internal fillet or gel is adapted for oral or parenteral administration for providing immunostimulation, immunomodulation, cancer treatment, diabetes treatment, anti-inflammatory action, or any combinations thereof.

13. The process of claim 1, wherein the anthraquinone rich aloe product may be combined with one or more medicinal preparations prepared from the internal fillet or the gel.

14. An anthraquinone rich aloe product generated by the process of claim 1.

15. An anthraquinone rich composition comprising:
a concentrated liquid, a lyophilized powder, a spray dried powder, or any combinations thereof comprising heat-treated and extracted mucilage from one or more rind parts from aloe leaves, wherein the composition comprises one or more preservatives.

16. The composition of claim 15, wherein the heat-treated and extracted mucilage is generated by a process comprising the steps of:
removing one or more rind parts from the aloe leaves comprising a top rind, a bottom rind, or both comprising the steps of:
filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer; and
separating the internal fillet or the gel layer from the rind parts;
removing a mucilage layer attached to the one or more rind parts by scraping, heat-treating the rind parts, or by any other mechanical extraction techniques;
heat-treating the extracted mucilage for a specified period of time; and
adding one or more preservatives to the heat-treated mucilage after cooling.

17. The composition of claim 16, wherein the process further comprises the steps of:
removing a base portion, a top portion and one or more spines along the margins of the aloe leaves; and
washing and cleaning the aloe leaves before filleting.

18. The composition of claim 16, wherein the process further comprise the step of collecting the mucilage accumulated on the filleting table, wherein the accumulated mucilage is added to the extracted mucilage prior to heat-treatment.

19. The composition of claim 16, wherein the step of filleting the aloe leaves is done by hand.

20. The composition of claim 15, wherein the internal fillet or gel is processed by one or more techniques to produce a medicinal composition for topical application, oral administration, or parenteral administration.

21. The composition of claim 15, wherein the extracted and heat-treated mucilage is enriched in one or more constituents comprising polysaccharides, organic acids, anthraquinones, and any combinations thereof.

22. The composition of claim 15, wherein the composition is used for a treatment of one or more conditions selected from constipation, malaria, cancer, or any combinations thereof.

23. The composition of claim 15, wherein the one or more preservatives are selected from the group consisting of benzoic acid, potassium sorbate, or other preservatives approved for human consumption.

24. A method of treating constipation, malaria, or any other parasitic infection in a human subject comprising the steps of:
identifying the human subject in need of treatment against the constipation, malaria or any other parasitic infection; and
administering a pharmaceutical composition comprising an anthraquinone rich aloe product to the human subject in an amount effective to treat the constipation, malaria or any other parasitic infection, wherein the composition comprises heat-treated and extracted mucilage from one or more rind parts from aloe leaves along with one or more preservatives.

25. The method of claim 24, wherein the pharmaceutical composition is made by a process comprising the steps of:
removing one or more rind parts from the aloe leaves comprising a top rind, a bottom rind, or both comprising the steps of:
filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer; and
separating the internal fillet or the gel layer from the rind parts;
removing a mucilage layer attached to the one or more rind parts by scraping, heat-treating the rind parts, or by any other mechanical extraction techniques;
heat-treating the extracted mucilage for a specified period of time; and
adding one or more preservatives to the heat-treated mucilage after cooling.

26. The method of claim 24, wherein the composition is adapted for topical, oral, or parenteral administration.

27. A pharmaceutical composition for treatment of constipation, malaria or any other parasitic infection in a human subject comprising:
an anthraquinone rich aloe product, wherein the composition comprises heat-treated and extracted mucilage from one or more rind parts from aloe leaves along with one or more preservatives.

28. A method of providing immunostimulation or immunomodulation, treating cancer or any combinations thereof in a human subject comprising the steps of:
identifying the human subject in need of immunostimulation, immunomodulation, treatment of the cancer, or any combinations thereof; and
administering a pharmaceutical composition comprising an anthraquinone rich aloe product to the human subject in an amount effective to provide immunostimulation, immunomodulation, treatment of the cancer, or any combinations thereof, wherein the composition comprises heat-treated and extracted mucilage from one or more rind parts from aloe leaves along with one or more preservatives and is administered to the human subject orally or parenterally.

29. The method of claim 28, wherein the pharmaceutical composition is made by a process comprising the steps of:
removing one or more rind parts from the aloe leaves comprising a top rind, a bottom rind, or both comprising the steps of:
filleting the aloe leaves on a filleting table using a knife or any other filleting equipment to by introducing the knife or the filleting equipment into a mucilage layer below the rind parts of the aloe leaves to remove the top rind, the bottom rind, or both from an internal fillet or a gel layer; and
separating the internal fillet or the gel layer from the rind parts;
removing a mucilage layer attached to the one or more rind parts by scraping, heat-treating the rind parts, or by any other mechanical extraction techniques;
heat-treating the extracted mucilage for a specified period of time; and
adding one or more preservatives to the heat-treated mucilage after cooling.

30. A pharmaceutical composition for providing immunostimulation or immunomodulation, treating cancer or any combinations thereof in a human subject comprising:
an anthraquinone rich aloe product, wherein the composition comprises heat-treated and extracted mucilage from one or more rind parts from aloe leaves along with one or more preservatives.
